(19) United States
(12) Patent Application Publication
(10) Pub. No.: US 2007/0198016 A1
(43) Pub. Date: Aug. 23, 2007

(54) Compression stabilizing spacers

(75) Inventors: Kerry Zang, Paradise Valley, AZ (US); Shaher A. Ahmad, Plano, TX (US)

Correspondence Address: BAKER BOTTS L.L.P., 2001 ROSS AVENUE, SUITE 600, DALLAS, TX, 75201-2980, US

(73) Assignee: OsteoMed, L.P.

(21) Appl. No.: 11/359,678

(22) Filed: Feb. 21, 2006

(51) Int. Cl. A61B 17/80; A61F 2/28

(52) U.S. Cl. 606/69; 623/17.11

(57) ABSTRACT

In accordance with an embodiment of the present invention, a joint fusion system includes a spacer configured to be positioned between a first bone of a joint and a second bone of the joint. The joint fusion system also includes a plate coupled to the spacer. The plate is configured to be coupled to the first bone and the second bone when the spacer is positioned between the first bone and the second bone to hold the spacer between the first bone and the second bone.

### TECHNICAL FIELD OF THE INVENTION

This invention relates generally to systems and methods of joint fusion and more specifically to compression stabilizing spacers.

### BACKGROUND

Arthrodesis is the fusion of joint. Two or more bones of a joint may be fused together to prevent relative movement between the bones of the joint. Joint fusion may be desirable when the bones of the joint have become degraded and no longer perform their function, or when the joint has become afflicted with severe arthritis.

Methods of joint fusion include fixing the bones of the joint to one another with screws and plates. Screws and plates provide fixation of the bones relative to one another, but may also result in a shortening of the physiological length of the joint. When the physiological length of the joint and surrounding bones has been shortened, other complications may result.

### SUMMARY

In accordance with an embodiment of the present invention a joint fusion system includes a spacer configured to be positioned between a first bone of a joint and a second bone of the joint. The joint fusion system also includes a plate coupled to the spacer. The plate is configured to be coupled to the first bone and the second bone when the spacer is positioned between the first bone and the second bone to hold the spacer between the first bone and the second bone.

In another embodiment of the present invention a spacer may include a cross section corresponding to a cross section of the first bone and a second bone. The spacer may also include a reservoir operable to hold a filler between the first bone and the second bone when the spacer is positioned between the first bone and the second bone. In certain embodiments, the filler may include a demineralized bone matrix and/or cancellous bone chips.

In yet another embodiment of the present invention a method of joint fusion may include placing a spacer between a first bone of a joint and a second bone of the joint. The method may also include coupling a plate to the spacer. The plate may be configured to be coupled to the first bone and the second bone when the spacer is positioned between the first bone and the second bone to hold the spacer between the first bone and the second bone.

Technical advantages of particular embodiments of the present invention may include preserving the physiological length of a joint following joint fusion by providing a spacer with a thickness corresponding to the removed or missing joint material. Rather than simply fixing the bones to each other using a plate and screws, a spacer may be inserted between the bones to hold the bones a specific distance from each other. The spacer may also be coupled to a plate and the plate may be coupled to each bone. The spacer may serve to stabilize the bones by providing a rigid abutment surface for each of the bones.

Another technical advantage of certain embodiments of the present invention may include an increase in the rate of healing and new bone growth, resulting in a faster and more stable joint fusion. To accomplish this, the area between the bones may be filled with a material that encourages bone growth. A spacer placed between the bones may include a reservoir that allows injection of a filler between the bones of the joint. The filler may encourage bone growth in the area between the bones. The reservoir may not only contain the bone growth filler, but may also allow new bone to grow within it, providing further fusion of the bones of the joint.

Still another technical advantage of certain embodiments of the present invention may include the ability to correct angulation deformities. A particular spacer may be wedge shaped, or otherwise shaped in a non-symmetrical manner such that the spacer may hold a first bone at a corrected orientation relative to a second bone. Joint degradation may include uneven wearing of the surfaces of the bones. Providing a spacer that is wider on one side than the other may allow correction of angulation deformities resulting from uneven wear or caused by other deformities.

Certain embodiments may provide all, some, or none of these advantages. Certain embodiments may provide one or more other advantages, one or more of which may be apparent to those skilled in the art from the figures, descriptions, and claims included herein.

### BRIEF DESCRIPTION OF THE DRAWINGS

To provide a more complete understanding of the present invention and the features and advantages thereof, reference is made to the following description, taken in conjunction with the accompanying drawings, in which:

FIG. 1A is a perspective view of a spacer, plate, and bones of a joint prior to fixing the spacer between the bones of the joint, in accordance with an embodiment of the present invention;

FIG. 1B is a perspective view of a spacer, plate and bones of a joint prior to fixing the spacer between the bones of the joint, in accordance with another embodiment of the present invention;

FIG. 2 is a top view of the assembled spacer and plate attached to the bones of the joint of FIG. 1A, in accordance with an embodiment of the present invention;

FIG. 3 illustrates a top view of a wedge shaped spacer installed in a joint in accordance with another embodiment of the present invention;

FIG. 4A illustrates a spacer designed for implantation into a relatively small first metatarsal-internal cuneiform joint in accordance with an embodiment of the present invention;

FIG. 4B illustrates a spacer designed for implantation into a relatively large first metatarsal-internal cuneiform joint in accordance with an embodiment of the present invention;

FIG. 5A illustrates a spacer designed for implantation into a relatively small navicular-internal cuneiform joint complex in accordance with a particular embodiment of the present invention;

FIG. 5B illustrates a spacer designed for implantation into a relatively large navicular-internal cuneiform joint complex in accordance with a particular embodiment of the present invention;

FIG. 6A illustrates a spacer designed for implantation into a relatively small calcanial elongation in accordance with a particular embodiment of the present invention;

FIG. 6B illustrates a spacer designed for implantation into a relatively large calcanial elongation in accordance with a particular embodiment of the present invention;

FIG. 7A illustrates a spacer designed for implantation into a relatively small calcanial-cuboid joint complex in accordance with a particular embodiment of the present invention;

FIG. 7B illustrates a spacer designed for implantation into a relatively large calcanial-cuboid joint complex in accordance with a particular embodiment of the present invention;

FIGS. 8A-E illustrate various plates for coupling a spacer between bones of a joint in accordance with particular embodiments of the present invention;

FIG. 9 illustrates a perspective view of cylindrical spacers and corresponding plates for implantation between bones of a joint in accordance with particular embodiments of the present invention;

FIG. 10A illustrates a spacer designed for implantation into a relatively small subtalar joint complex in accordance with a particular embodiment of the present invention;

FIG. 10B illustrates a spacer designed for implantation into a relatively large subtalar joint complex in accordance with a particular embodiment of the present invention;

FIG. 11 illustrates a top view of the spacer of FIG. 10B in accordance with an embodiment of the present invention; and

FIG. 12 illustrates a threaded spacer for implantation into a relatively large subtalar joint complex in accordance with an embodiment of the present invention.

### DESCRIPTION OF EXAMPLE EMBODIMENTS

Embodiments of the present invention include spacers for maintaining a desired distance between bones of a joint when the joint is being fused. The spacers may be coupled to the bones using plates and/or bone screws. In particular embodiments, the spacers may include reservoirs to allow injection of a filler material to stimulate bone growth between the bones of the joint.

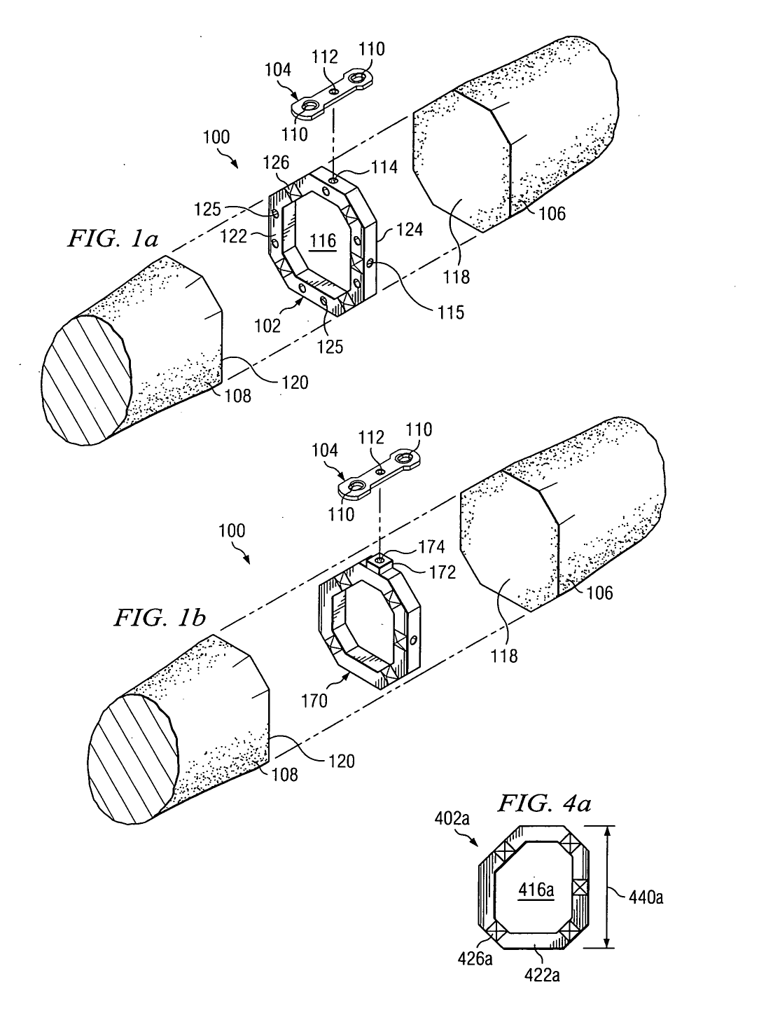

FIG. 1A is an exploded perspective view of a joint fusion system 100. Joint fusion system 100 includes a spacer 102 and a plate 104 for coupling a first bone 106 of a joint with a second bone 108 of a joint. Bones 106 and 108 may be bones of a joint that has become damaged making it desirable to fuse the joint. Spacer 102 may be inserted between bones 106 and 108. Plate 104 may then be coupled to spacer 102 and bones 106 and 108. Plate 104 may thereby securely couple spacer 102, bone 106, and bone 108. Spacer 102 may provide and maintain a desired amount of spacing between bones 106 and 108. In a particular embodiment, spacer 102 may be designed to maintain a spacing between bones 106 and 108 that existed prior to the joint becoming damaged. Spacer 102 and spacers in accordance with other embodiments such as those further discussed below may be manufactured from titanium, PEEK, surgical stainless steel, bioresorbable material, bone substitute or any other suitable material.

Plate 104 may be coupled to spacer 102 by a screw or other appropriate coupling method. In the embodiment illustrated, a screw may be passed through hole 112 in plate 104 and be threaded into threaded hole 114 on spacer 102. Likewise, plate 104 may be coupled to bones 106 and 108 using bone screws or other appropriate method. In the illustrated embodiment, holes 110 include a wedge shaped compression feature (see FIGS. 8A to 8E) that forces bones 106 and 108 into closer proximity as screws are inserted through holes 110. The design of holes 110 may result in bones 106 and 108 exerting a compressive force on spacer 102 when appropriate screws are used to couple plate 104 to bones 106 and 108.

Spacer 102 also includes a reservoir 116. Reservoir 116 may allow the introduction of a filler material for stimulating bone growth between bones 106 and 108. In particular embodiments, the filler material for stimulating bone growth may include demineralized bone matrix, cancellous bone chips, bone substitute, bone cement, infection preventative, biologics, antibiotics, and bone morphogenic proteins. The filler material may be injected through hole 115 after bones 106 and 108 have been coupled with spacer 102. The filler material may stimulate bone growth in the volume between bones 106 and 108 defined by reservoir 116. The new bone that grows between bones 106 and 108 will aid in fusing bones 106 and 108. Injecting a filler material into reservoir 116 may also speed the healing and recovery of the operative site.

Spacer 102 may also include a first face 122 for interfacing with end 120 of bone 108. A corresponding second face 124 (not visible) may intersect with end 118 of bone 106. The shape of spacer 102 and faces 122 and 124 may correspond with the shapes of ends 120 and 118. That is, a cross section of spacer 102 may generally correspond to a cross section of ends 118 and 120, or the joint in which spacer 102 is utilized. A correspondence of cross sections between spacer 102 and the joint provides a smooth transition between bones 106 and 108 and reduces the chance of soft tissue irritation that may result from spacer 102 protruding beyond the edges of bones 106 and 108.

First face 122 and second face 124 may optionally include protuberances 126. Protuberances 126 may interface with ends 120 and 118 to stabilize spacer 102 within the joints of bones 106 and 108. Protuberances 126 may prevent rotation or slippage of spacer 102 relative to bones 106 and 108. Furthermore, as bones 106 and 108 are compressed on either side of spacer 102, the protuberances 126 may penetrate the ends 118 and 120 of bones 106 and 108 and further stabilize spacer 102. A specific number and arrangement of protuberances 126 is not essential to the invention, and practically any number arrangement of protuberances 126 may be utilized. In particular embodiments, protuberances 126 may not be present and an alternative stabilizing surface, such as knurling of face 122, may be utilized. In other embodiments, faces 122 and 124 may interface with ends 120 and 118 without any extra stabilizing mechanisms.

In the illustrated embodiment, spacer 102 also includes a plurality of holes 125 that extend from first face 122 to second face 124. Holes 125 allow bone to grow through the spacer (e.g., in a similar manner to the growth of bone in reservoir 116) to further strengthen the joint. It should be understood that spacers in other embodiments, such as those discussed below, may also include similar holes even though they may not be specifically illustrated herein.

FIG. 1B is an exploded perspective view of a joint fusion system in accordance with another embodiment. The joint fusion system of FIG. 1B includes a spacer 170 similar to spacer 102 of FIG. 1A except that spacer 170 includes a smaller total circumference than that of spacer 102. Thus, the outer edges of spacer 170 do not extend to the outer edges of the bones at the joint (e.g., bones 106 and 108 of FIG. 1A). This allows bone to form at the joint around the outer edges of spacer 170. Spacer 170 includes protrusion 172 that extends to the edge of the joint bones (for example, as far as spacer 102 extends) and that comprises a hole 174 similar to hole 114 of spacer 102 for coupling spacer 170 to a plate.

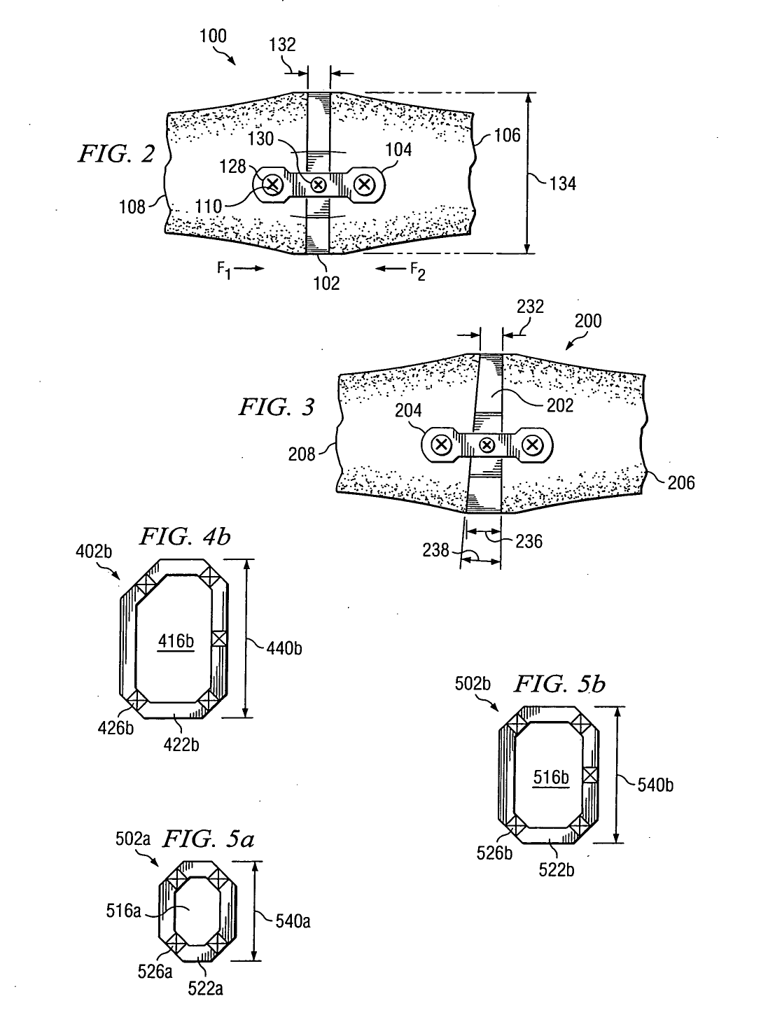

FIG. 2 illustrates a top view of joint fusion system 100 as assembled. Spacer 102 has been placed between bones 106 and 108. Plate 104 has been coupled to spacer 102 by a screw 130 through hole 112. Furthermore, plate 104 has been coupled to bones 106 and 108 using bone screws 128 through holes 110. As screw 128 is inserted into bone 108, screw 128 slides within the wedge shaped portion of hole 110 (see FIGS. 8A to 8E) and creates a force on bone 108 directing bone 108 in the direction of arrow F1. Likewise, as screw 128 is inserted into bone 106, screw 128 slides within the wedge shaped portion of hole 110 (see FIGS. 8A to 8E) and creates a force on bone 106 in the direction of arrow F2. Force F1 acting on bone 108 and force F2 acting on bone 106 compress the bones on spacer 102. The compression of bones 106 and 108 minimizes the gaps between bones 106, 108, and spacer 102 and provides the proper spacing between bones 106 and 108. The proper spacing between bones 106 and 108 corresponds to a thickness 132 of spacer 102. Spacer 102 may be designed with a thickness 132 corresponding to the desired distance that bones 106 and 108 should be held apart. Thickness 132 may be designed to maintain a physiological length of the joint between bones 106 and 108 prior to fusion or prior to the joint becoming damaged.

Spacer 102 also has a width 134 corresponding to a width of the joint between bones 106 and 108. Width 134 of spacer 102 may be selected such that spacer 102 does not protrude beyond the edges of bones 106 or 108. As discussed above, sizing spacers such that they do not protrude beyond the edges of bones may reduce the chance of soft tissue irritation. In some embodiments such as the spacer illustrated and described with respect to FIG. 1B, the width of a spacer may not even extend to the edges of bones 106 or 106. This may allow bone to form around the exterior of the spacer and may provide greater support for bones 106 and 108.

FIG. 3 illustrates a bone fusion system 200. Bone fusion system 200 includes a spacer 202 coupled to a plate 204. Plate 204 couples spacer 202 with bones 206 and 208. Like spacer 102 discussed above, spacer 202 may maintain a desired spacing between bones 206 and 208. In addition to the functionality described above with respect to spacer 102, spacer 202 may also correct angulation deformities between bones 206 and 208. A width 232 on one side of spacer 202 is less than a width 236 on the opposite side of spacer 202. In the illustrated embodiment, spacer 202 is generally wedge shaped. An angle 238 of spacer 202 may correspond to a desired angle of correction between bones 206 and 208. With this design, spacer 202 may change an orientation of bone 208 relative to bone 206. The angle 238 may be chosen based on the amount of correction required to correct the angulation deformity between bone 206 and bone 208.

FIGS. 4A and 4B illustrate specific embodiments of spacers such as spacers 102 and/or 202 described above. Spacers 402A and 402B illustrate two embodiments of spacers that may be implanted in the first metatarsal cuneiform joint. In some embodiments, spacers 402A and 402B may comprise a wedge shape similar to spacer 202 described above. As illustrated, spacer 402A is relatively smaller than spacer 402B. While a general shape of spacer 402A may correspond to a general shape of spacer 402B, a height 440A may be less than a height 440B. In this manner, spacer 402A may be designed to fit a relatively smaller joint than spacer 402B. While only a variance in the height 440 has been illustrated, any appropriate dimension of spacers 402 may be altered to suit a specific joint. Spacers 402A and 402B may include reservoirs 416, faces 422, and optional protuberances 426 having similar functionality as reservoir 116, face 122, and protuberances 126, respectively, described above.

FIGS. 5A and 5B illustrate further specific embodiments of spacers such as spacers 102 and/or 202 described above. Spacers 502A and 502B illustrate two embodiments of spacers that may be implanted in the navicular cuneiform joint complex. In some embodiments, spacers 502A and 502B may comprise a wedge shape similar to spacer 202 described above. As illustrated, spacer 502A is relatively smaller than spacer 502B. While a general shape of spacer 502A may correspond to a general shape of spacer 502B, a height 540A may be less than a height 540B. In this manner, spacer 502A may be designed to fit a relatively smaller joint than spacer 502B. While only a variance in the height 540 has been illustrated, any appropriate dimension of spacers 502 may be altered to suit a specific joint. Spacers 502A and 502B may include reservoirs 516, faces 522, and optional protuberances 526 having similar functionality as reservoir 116, face 122, and protuberances 126, respectively, described above.

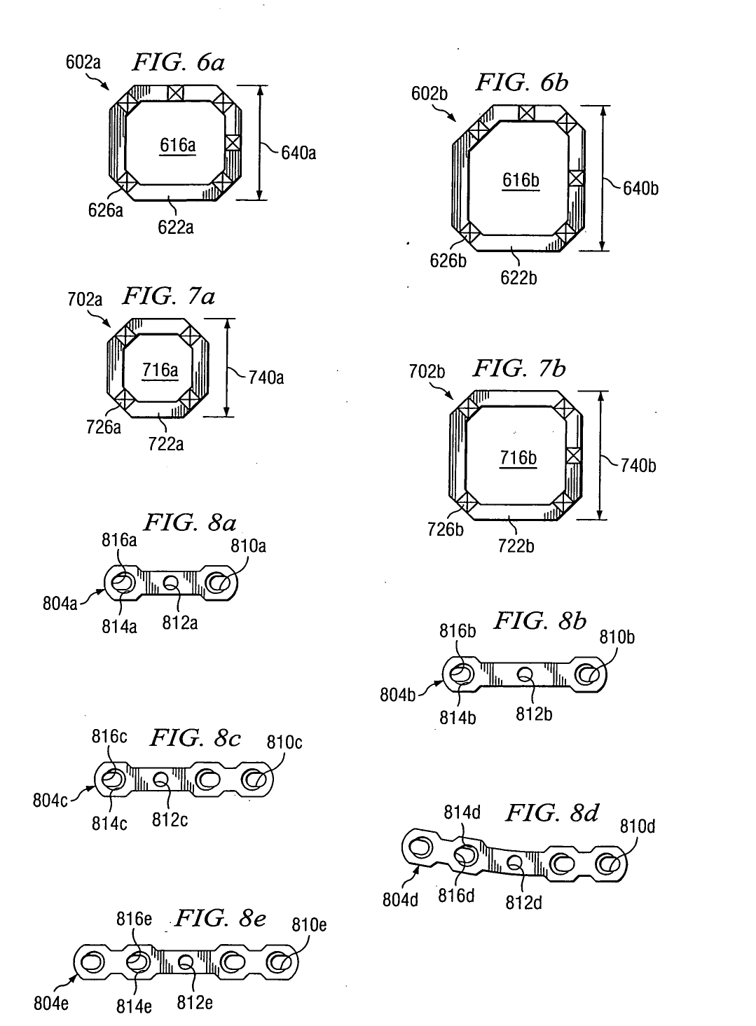

FIGS. 6A and 6B illustrate further specific embodiments of spacers such as spacers 102 and/or 202 described above. Spacers 602A and 602B illustrate two embodiments of spacers that may utilized for Evans calcaneal osteotomy, or calcaneal elongation. In some embodiments, spacers 602A and 602B may comprise a wedge shape similar to spacer 202 described above. As illustrated, spacer 602A is relatively smaller than spacer 602B. While a general shape of spacer 602A may correspond to a general shape of spacer 602B, a height 640A may be less than a height 640B. In this manner, spacer 602A may be designed to fit a relatively smaller joint than spacer 602B. While only a variance in the height 640 has been illustrated, any appropriate dimension of spacers 602 may be altered to suit a specific joint. Spacers 602A and 602B may include reservoirs 616, faces 622, and optional protuberances 626 having similar functionality as reservoir 116, face 122, and protuberances 126, respectively, described above.

FIGS. 7A and 7B illustrate further specific embodiments of spacers such as spacers 102 and/or 202 described above. Spacers 702A and 702B illustrate two embodiments of spacers that may be implanted in the calcaneal-cuboid joint complex. As illustrated, spacer 702A is relatively smaller than spacer 702B. While a general shape of spacer 702A may correspond to a general shape of spacer 702B, a height 740A may be less than a height 740B. In this manner, spacer 702A may be designed to fit a relatively smaller joint than spacer 702B. While only a variance in the height 740 has been illustrated, any appropriate dimension of spacers 702 may be altered to suit a specific joint. Spacers 702A and 702B may include reservoirs 716, faces 722, and optional protuberances 726 having similar functionality as reservoir 116, face 122, and protuberances 126, respectively, described above.

While various embodiments of spacers designed for the first metatarsal-internal cuneiform joint, the navicular-internal cuneiform joint complex, Evans calcaneal osteotomy, and the calcaneal-cuboid joint complex have been illustrated and described above, spacers embodying the teachings of the present invention may be used to fuse practically any joint. For example, spacers may also be designed for the talo-navicular joint complex or the subtalar joint complex. As a further example, the above described spacers are designed to fuse joints of the human foot and ankle, however, the above described system and methods may be equally applicable to joints of the hand, wrist, or any other suitable joint were fusion is desirable or necessary.

FIGS. 8A-8E illustrate a variety of plates in accordance with particular embodiments of the present invention. Plates 804 include holes 810 and 812. Holes 812 may be utilized to couple the plate to the spacers as described above. Holes 810 may be used to couple the plates 804 to various bones. Holes 810 include a pilot section 816 and a wedge section 814. During insertion, a screw may initially be positioned at a center of pilot section 816. As the screw is screwed into the hole, the screw may move laterally relative to the plate towards wedge section 814 and, when completely inserted into the target bone, may reside at a center of wedge portion 814. An underside of a head of a screw used to couple plate 804 to a bone may correspond in shape to wedge shape 814. Thus, as the screw is inserted into pilot section 816, wedge portion 814 will draw in the screw such that the screw head will come to reside in wedge portion 814. This movement of the screw relative to the portions of hole 810 creates a compressive force on the bones that compresses the bones on either side of a spacer.

FIGS. 8A-E illustrate a variety of embodiments of plates 804. A particular plate 804 may be chosen based on a desired amount of fixation, a width a spacer 102, or an orientation of the bones on either side of spacer 102. In addition to the various embodiments illustrated, many other plates may be imagined by one of skill in the art. Alternative embodiments of plates may include any suitable number of holes 810 and may be practically any shape to correspond to shapes of bones or shapes of joints. Furthermore, although only one plate has been illustrated as coupling a spacer to respective bones, potentially any number of plates may be used with a single spacer to achieve a desired level of fixation and support for the spacer. For example, some embodiments may include a spacer coupled to plates on both a top and a bottom of a joint.

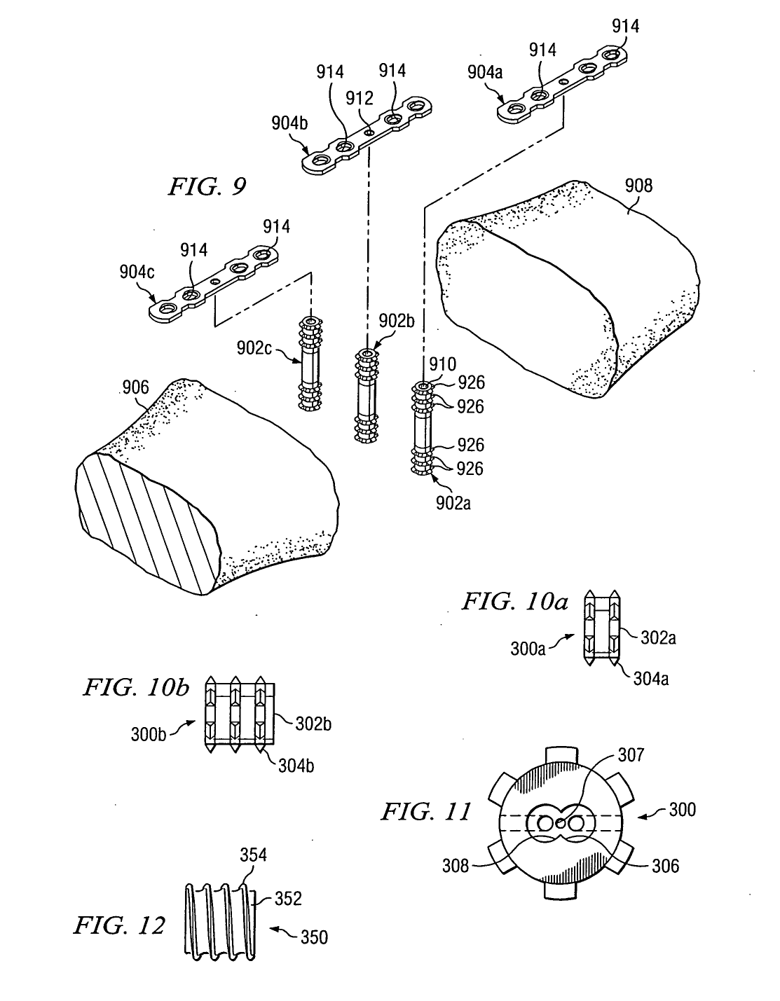

FIG. 9 illustrates a joint fusion system 900. Joint fusion system 900 includes one or more spacers 902 coupled to bones 906 and 908 by a plate 904. In the illustrated embodiment, three spacers, 902A, 902B, and 902C have been placed between bones 906 and 908. Spacers 902 may have a width corresponding to a desired distance to be maintained between bones 906 and 908. Spacers 902 may also include protuberances 926, which may have similar functionality as protuberances 126 described above. In this embodiment, spacers 902 each have a generally cylindrical shape.

Plates 904 may be coupled to spacers 902 using a screw or other appropriate fixing method passing through hole 912 of plate 904. Plates 904 may be coupled to bones 906 and 908 using holes 914. Holes 914 may be similar to holes 810 described above, and may impart a compressive force on bones 906 and 908 towards spacers 902.

While three spacers 902 have been illustrated between bones 906 and 908, practically any number of spacers 902 may be utilized as appropriate for a particular joint between bones. Additionally, each spacer 902 has been illustrated with a corresponding plate 904, however, multiple plates may be used with each spacer 902, or a single plate may be used to couple one or more of spacers 902 to bones 906 and 908.

FIGS. 10A and 10B illustrate cylindrical spacers for implantation into a subtalar joint to fuse the talus and calcaneus bones. In the illustrated embodiment, spacers 300 include generally cylindrical bodies 302. Other embodiments may include spacers having tapered, round bodies such that one end of the spacer is wider than the opposite end of the spacer. Arranged around the exterior of cylindrical bodies 302 are protuberances 304. Protuberances 304 may have similar functionality to protuberances 126, described above. Spacer 300A may be designed to be implanted in a relatively small subtalar joint while spacer 300B may be designed to be implanted in a relatively larger subtalar joint. The sizes of spacers 300A and 300B may correspond to a desired distance to maintain between the talus and calcaneus bones.

FIG. 11 illustrates a top view of spacers 300. As illustrated in FIG. 11, spacers 300 include two angled holes 306 and 308 and a center hole 307. Holes 306 and 308 may receive bone screws. A bone screw inserted through hole 306 may couple the talus bone to spacer 300. A screw inserted through hole 308 may couple the calcaneus bone to spacer 300. Insertion of screws through holes 306 and 308 will result in a fusion of the joint between the talus and calcaneus bones. Center hole 307 may be used for the insertion of filler, such as demineralized bone matrix, cancellous bone chips, bone substitute, bone cement, infection preventative, biologics, antibiotics, and bone morphogenic proteins.

FIG. 12 illustrates another embodiment of a spacer for implantation into the subtalar joint. Spacer 350 includes a generally cylindrical body 352 and a plurality of threads 354. Threads 354 may replace protuberances 304 and provide a stabilizing function as described above with reference to protuberances 126. In addition to the stabilizing function, threads 354 may increase the ease of installation of spacer 350 into the subtalar joint. After implantation of spacer 350, spacer 350 may be coupled to the talus and calcaneus bones using bone screws as described above with reference to FIG. 11.

Although the present invention has been described with several embodiments, a number of changes, substitutions, variations, alterations, and modifications may be suggested to one skilled in the art, and it is intended that the invention encompass all such changes, substitutions, variations, alterations, and modifications as fall within the spirit and scope of the appended claims.

What is claimed is:

1. A joint fusion system, comprising:
a spacer configured to be positioned between a first bone of a joint and a second bone of the joint; and
a plate coupled to the spacer, the plate configured to be coupled to the first bone and the second bone when the spacer is positioned between the first bone and the second bone to hold the spacer between the first bone and the second bone.

2. The system of claim 1, wherein the spacer comprises a cross section corresponding to a cross section of the first bone and the second bone.

3. The system of claim 1, wherein the spacer defines a reservoir operable to hold a filler between the first bone and the second bone when the spacer is positioned between the first bone and the second bone.

4. The system of claim 3, wherein an exterior surface of the spacer defines a hole configured to receive the filler for insertion into the reservoir.

5. The system of claim 3, wherein the filler comprises one or more materials selected from the group consisting of: demineralized bone matrix, cancellous bone chips, bone substitute, bone cement, infection preventative, biologics, antibiotics, and bone morphogenic proteins.

6. The system of claim 1, wherein the spacer comprises a first face and a second face generally opposite the first face, the first face comprising at least one protuberance operable to interface with the first bone to stabilize the spacer relative to the first bone.

7. The system of claim 1:
wherein the plate defines first and second holes; and
further comprising first and second screws, the first screw positioned through the first hole of the plate to couple the plate to the first bone and the second screw positioned through the second hole of the plate to couple the plate to the second bone.

8. The system of claim 7, wherein the first hole includes a wedge shaped portion that directs a head of the first screw toward the joint such that the first screw exerts a compressive force on the joint to push the first and second bones together when the first screw is positioned through the first hole.

9. The system of claim 1, wherein the spacer comprises a first face and a second face generally opposite the first face, and wherein the first face is approximately parallel to the second face.

10. The system of claim 1, wherein the spacer comprises a first face and a second face generally opposite the first face, and wherein the first face is not parallel to the second face such that a thickness of the spacer varies from a first end of the spacer to a second end of the spacer.

11. The system of claim 1, wherein the spacer comprises a first face and a second face generally opposite the first face, the first face shaped to correspond to a shape of an end of the first bone.

12. The system of claim 1, wherein the spacer comprises a thickness corresponding to a desired distance to be maintained between the first and second bones when the spacer is positioned between the first and second bones.

13. The system of claim 1, wherein the spacer comprises a shape corresponding to a desired orientation to be maintained between the first and second bones when the spacer is positioned between the first and second bones.

14. A spacer, comprising:
a cross section corresponding to a cross section of a first bone and a second bone; and
a reservoir operable to hold a filler between the first bone and the second bone when the spacer is positioned between the first bone and the second bone.

15. The system of claim 14, wherein the filler comprises one or more materials selected from the group consisting of: demineralized bone matrix, cancellous bone chips, bone substitute, bone cement, infection preventative, biologics, antibiotics, and bone morphogenic proteins.

16. The spacer of claim 14, further comprising a first face and a second face generally opposite the first face, the first face comprising at least one protuberance operable to interface with the first bone to stabilize the spacer relative to the first bone.

17. The spacer of claim 14, wherein the spacer comprises a first face and a second face generally opposite the first face, and wherein the first face is approximately parallel to the second face.

18. The spacer of claim 14, further comprising a first face and a second face generally opposite the first face, and wherein the first face is not parallel to the second face such that a thickness of the spacer varies from a first end of the spacer to a second end of the spacer.

19. The spacer of claim 14, further comprising a first face and a second face generally opposite the first face, the first face shaped to correspond to a shape of an end of the first bone.

20. The spacer of claim 14, further comprising a thickness corresponding to a desired distance to be maintained between the first and second bones when the spacer is positioned between the first and second bones.

21. The spacer of claim 14, further comprising a shape corresponding to a desired orientation to be maintained between the first and second bones when the spacer is positioned between the first and second bones.

22. A method of joint fusion, comprising:
placing a spacer between a first bone of a joint and a second bone of the joint; and
coupling a plate to the spacer, the plate configured to be coupled to the first bone and the second bone when the spacer is positioned between the first bone and the second bone to hold the spacer between the first bone and the second bone.

23. The method of claim 22, wherein the spacer comprises a cross section corresponding to a cross section of the first bone and the second bone.

24. The method of claim 22, further comprising injecting a filler into a reservoir of the spacer, the reservoir operable to hold the filler between the first bone and the second bone when the spacer is positioned between the first bone and the second bone.

25. The method of claim 24, wherein the filler comprises one or more materials selected from the group consisting of: demineralized bone matrix, cancellous bone chips, bone substitute, bone cement, infection preventative, biologics, antibiotics, and bone morphogenic proteins.

26. The method of claim 22, further comprising stabilizing the spacer relative to the first bone with a protuberance operable to interface with the first bone, the protuberance being disposed on a first face of the spacer.

27. The method of claim 22, further comprising:
positioning a first screw through a first hole of the plate to couple the plate to the first bone; and
positioning a second screw through a second hole of the plate to couple the plate to the second bone.

28. The method of claim 27, wherein the first hole includes a wedge shaped portion that directs a head of the first screw toward the joint such that the first screw exerts a compressive force on the joint to push the first and second bones together when the first screw is positioned through the first hole.

29. The method of claim 22, wherein the spacer comprises a first face and a second face generally opposite the first face, and wherein the first face is approximately parallel to the second face.

30. The method of claim 22, wherein the spacer comprises a first face and a second face generally opposite the first face, and wherein the first face is not parallel to the second face such that a thickness of the spacer varies from a first end of the spacer to a second end of the spacer.

31. The method of claim 22, wherein the spacer comprises a first face and a second face generally opposite the first face, the first face shaped to correspond to a shape of an end of the first bone.

32. The method of claim 22, wherein the spacer comprises a thickness corresponding to a desired distance to be maintained between the first and second bones when the spacer is positioned between the first and second bones.

33. The method of claim 22, wherein the spacer comprises a shape corresponding to a desired orientation to be maintained between the first and second bones when the spacer is positioned between the first and second bones.

34. A subtalar spacer, comprising:
a generally cylindrical body comprising a diameter corresponding to an amount of space to be maintained between a first bone and a second bone;
the generally cylindrical body defining a first screw hole and a second screw hole;
the first screw hole configured to accept a first bone screw to couple the cylindrical body to the first bone, and the second screw hole configured to accept a second bone screw to couple the cylindrical body to the second bone.

35. The subtalar spacer of claim 34, wherein the first bone is the talus bone and the second bone is the calcaneus bone.

36. The subtalar spacer of claim 34, wherein the cylindrical body comprises a plurality of threads operable to guide the subtalar spacer during implantation between the first and second bones.
